(19) United States
(12) Patent Application Publication
(10) Pub. No.: US 2007/0224254 A1
(43) Pub. Date: Sep. 27, 2007

(54) Transdermal delivery patch

(75) Inventors: Chien-Chih Yu, Yung-Kang City (TW); Ya-I Huang, Tainan City (TW); Thau-Ming Cham, Kaohsiung City (TW); Feng-O Lu, Yung-Kang City (TW); Tzu-Yin Chen, Tainan City (TW); Tsai-Wen Yu, Nei-Pu Tsun (TW)

Correspondence Address: MERCHANT & GOULD PC, P.O. BOX 2903, MINNEAPOLIS, MN, 55402-0903, US

(73) Assignee: SHENG CHUN TANG PHARMACEUTICAL INDUSTRIAL CO., LTD., Yung-Kang City (TW)

(21) Appl. No.: 11/726,909

(22) Filed: Mar. 22, 2007

(30) Foreign Application Priority Data
Mar. 23, 2006 (TW) 095110053

(51) Int. Cl. A61K 36/906; A61F 13/02; B01J 13/02; A61K 9/70

(52) U.S. Cl. 424/449; 424/478; 424/725; 424/756; 427/213.3; 514/692; 604/304

(57) ABSTRACT

A transdermal delivery patch includes: a matrix; a volatile component; and a plurality of microcapsules dispersed in the matrix. Each of the microcapsules has an enclosing wall enclosing the volatile component therein. A method for preparing the transdermal delivery patch includes: mixing a first gel, a second gel with a charge opposite to that of the first gel, and a volatile component so as to form microcapsules enclosing the volatile component, each of the microcapsules having an enclosing wall that is composed of the first and second gels; dispersing the microcapsules in a matrix so as to form a medicinal dressing; and coating the medicinal dressing on a backing substrate.

### CROSS-REFERENCE TO RELATED APPLICATION

This application claims priority of Taiwanese application no. 095110053, filed on Mar. 23, 2006.

### BACKGROUND OF THE INVENTION

1. Field of the Invention

This invention relates to a transdermal delivery patch, more particularly to a transdermal matrix patch including a plurality of microcapsules which enclose a volatile component therein.

2. Description of the Related Art

A transdermal matrix patch usually includes a backing substrate and a medicinal dressing coated on the backing substrate and containing a drug. In order to relieve pain, a volatile drug and/or a refreshing and cooling volatile component, such as menthol, methyl salicylate, or aromatic essential oil extracted from plants, is included in the medicinal dressing. However, during storage, the volatile drug or volatile component is likely to volatize or be deteriorated by external environment, e.g., oxygen, carbon dioxido, water, or light, so that effectivity of the transdermal matrix patch on pain-relief is reduced.

### SUMMARY OF THE INVENTION

Therefore, there is a need in the art to provide a transdermal delivery patch that can overcome the aforesaid drawback of the prior art.

According to one aspect of this invention, a transdermal delivery patch includes: a matrix; a volatile component; and a plurality of microcapsules dispersed in the matrix. Each of the microcapsules has an enclosing wall enclosing the volatile component therein.

According to another aspect of this invention, a method for preparing a transdermal delivery patch includes: mixing a first gel, a second gel with a charge opposite to that of the first gel, and a volatile component so as to form microcapsules enclosing the volatile component, each of the microcapsules having an enclosing wall that is composed of the first and second gels; dispersing the microcapsules in a matrix so as to form a dressing; and coating the dressing on a backing substrate.

### BRIEF DESCRIPTION OF THE DRAWINGS

Other features and advantages of the present invention will become apparent in the following detailed description of the preferred embodiment of this invention, with reference to the accompanying drawings, in which;

FIG. 1 is a fragmentary schematic cross-sectional view of the preferred embodiment of a transdermal delivery patch according to this invention;

FIG. 2 is a fragmentary enlarged cross-sectional view of the preferred embodiment;

FIG. 3 is a flow chart illustrating a method of the preferred embodiment for preparing the transdermal delivery patch shown in FIG. 1;

FIG. 4 is a flow chart illustrating consecutive steps for producing microcapsules included in the transdermal delivery patch shown in FIG. 1;

FIG. 5 is a plot showing dissolution rates of menthol for a traditional hydrophilic transdermal patch and a hydrophilic transdermal matrix patch of this invention at 25° C.;

FIG. 6 is a plot showing dissolution rates of methyl salicylate for a traditional hydrophilic transdermal patch and a hydrophilic transdermal matrix patch of this invention at 25° C.;

FIG. 7 is a plot showing dissolution rates of menthol for a traditional hydrophobic transdermal patch and a hydrophobic transdermal matrix patch of this invention at 25° C.;

is FIG. 8 is a plot showing dissolution rates of methyl salicylate for a traditional hydrophobic transdermal patch and a hydrophobic transdermal matrix patch of this invention at 25° C.;

FIG. 9 is a plot showing dissolution rates of menthol for a hydrophilic transdermal matrix patch of this invention at 25° C. and 37° C.;

FIG. 10 is a plot showing dissolution rates of methyl salicylate for a hydrophilic transdermal matrix patch of this invention at 25° C. and 37° C.;

FIG. 11 is a plot showing dissolution rates of menthol for a hydrophobic transdermal matrix patch of this invention at 25° C. and 37° C.; and

FIG. 12 is a plot showing dissolution rates of methyl salicylate for a hydrophobic transdermal matrix patch of this invention at 25° C. and 37° C.

### DETAILED DESCRIPTION OF THE PREFERRED EMBODIMENT

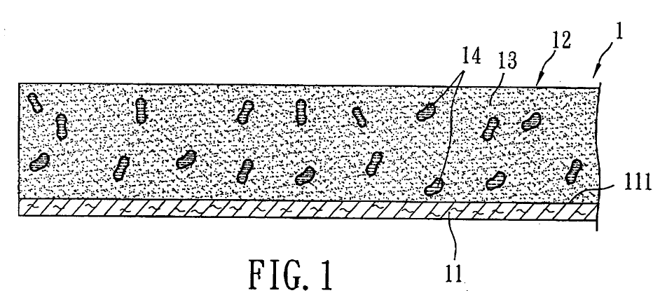

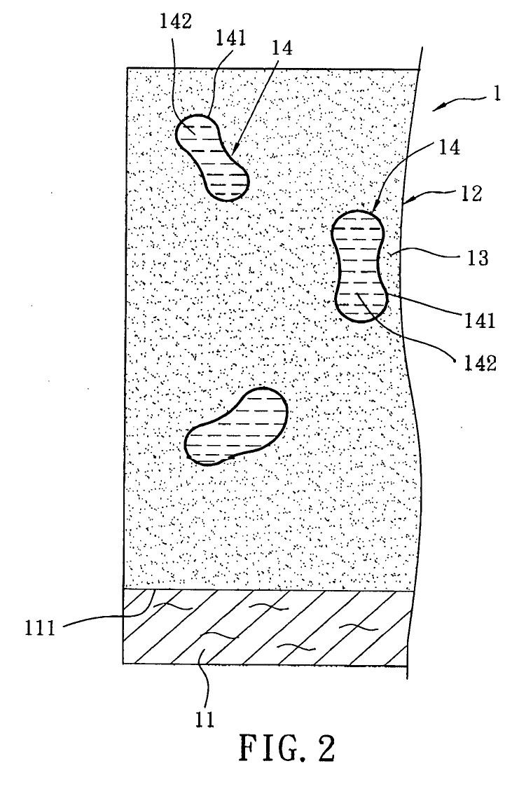

Referring to FIGS. 1 and 2, the preferred embodiment of a transdermal delivery patch 1 according to the present invention is shown to include a backing substrate 11 and a medicinal dressing 12 bonded to the backing substrate 11. The backing substrate 11 is made from a fabric material or a gas permeable rubber material. The medicinal dressing 12 includes: a matrix 13 bonded to a surface 111 of the backing substrate 11; a volatile component 142; and a plurality of microcapsules 14 dispersed in the matrix 13. Each of the microcapsules 14 has an enclosing wall 141 is enclosing the volatile component 142 therein, and has a granular size ranging from 10 to 30 μm. The volatile component 142 can be efficiently maintained in the microcapsules 14 during storage, and can be released from the microcapsules 14 by penetrating through the enclosing walls 141 upon application to a user. The dissolution rate of the volatile component 142 from the microcapsules 14 is dependent on temperature (will be described in the following context).

The matrix 13 of this invention includes an aqueous phase composition, an oil phase composition containing a volatile component similar to or different from the volatile component 142 enclosed in the microcapsules 14, and a solvent for dissolving the volatile component, an emulsifying agent, and a crosslinking agent.

The aqueous phase composition contains water, and a hydrophilic material including at least one of following components as an adhesive agent/thickening agent, i.e., sodium polyacrylate, potassium polyacrylate, ammonium polyacrylate, monoethanolamine polyacrylate, diethanolamine polyacrylate, triethanolamine polyacrylate, carboxylmethyl cellulose sodium, carboxyl methyl starch, Tragacanth, polyvinyl alcohol, polycaroboxyethylene, polyethylene pyrrolidone, polyacrylic acid, polyethylene glycol, acacia, hydroxypropyl cellulose, hydroxyethyl cellulose, hydroxymethyl cellulose, gelatin, and agar. An example for the adhesive agent/thickening agent used in the preferred embodiment of this invention is Carbopol 941 (i.e., Carbomer® 941).

Examples of the solvent for the oil phase composition include at least one of paraffin wax, silicon, isobutyl phthalate, diethyl phthalate, isopropyl phthalate, dibutyl phthalate, diethyl hexanedioate, diethyl octanedioate, or diethyl decanedioate.

Examples of the emulsifying agent include at least one of lauryl sodium sulfate; tween group (e.g., polyoxyethylene sorbitan monolaurate, polyoxyethylene sorbitan monopalmitate, polyoxyethylene sorbitan monostearate, polyoxyethylene sorbitan tristearate, polyoxyethylene sorbitan monoleate, and polyoxyethylene sorbitan trioleate); span group (e.g., sorbitan monolaurate, sorbitan monopalmitate, sorbitan monostearate, sorbitan tristearate, sorbitan monoleate, sorbitan sesquioleate, and sorbitan trioleate); and a monovalence soap formed of stearate and sodium hydroxide, potassium hydroxide, or ammonium hydroxide.

Examples of the crosslinking agent include at least one of aluminium hydroxide, aluminium chloride, aluminium potassium sulfate, aluminium carbonate, aluminium acetate, aluminium nitrate, magnesium hydroxide, magnesium aluminate, magnesium nitrate, calcium chloride.

In addition to the aqueous phase composition, the solvent, the emulsifying agent, and the crosslinking agent, the matrix 13 of the transdermal delivery patch 1 of this invention further includes a moisturizing agent and a filling agent. Examples of the moisturizing agent include at least one of glycerol, mannitol, propylene glycol, and sorbitol. The filling agent includes, for example, a penetration enhancer (e.g., ethanol, propylene glycol, aliphatic ester, dimethyl sulfone, oleic acid, linoleic acid, lauryl alcohol, azone (1-dodecylhexahydro-2H-azepin-2-one), lauryl sodium sulfate, Tween 80 (polyethylenated sorbitan monooleate, urea, salicylic acid, N-methyl-pyrrolidone, N-ethyl-pyrrolidone, menthol, 2-camphanone, limonene, or cajuputole).

When the volatile component 142 is required to be slowly released from the transdermal delivery patch 1 of this invention, the aforesaid hydrophilic adhesive agent/thickening agent employed for forming the aqueous phase composition is substituted with a hydrophobic material including at least one of synthetic rubber, natural rubber, pine resin, and resin. The transdermal delivery patch 1 including the matrix 12 formed of the hydrophobic material is referred as a hydrophobic transdermal delivery patch. On the contrary, the transdermal delivery patch 1 including the matrix 12 formed of the hydrophilic material is referred as a hydrophilic transdermal delivery patch.

The volatile component 142 enclosed in the microcapsules 14 and in the matrix 12 is, for example, menthol, methylsalicylate, and aromatic essential oil extracted from a plant, such as Rhizoma Zingiberis Recens, Fructus Foeniculi, and Flos Caryphylli.

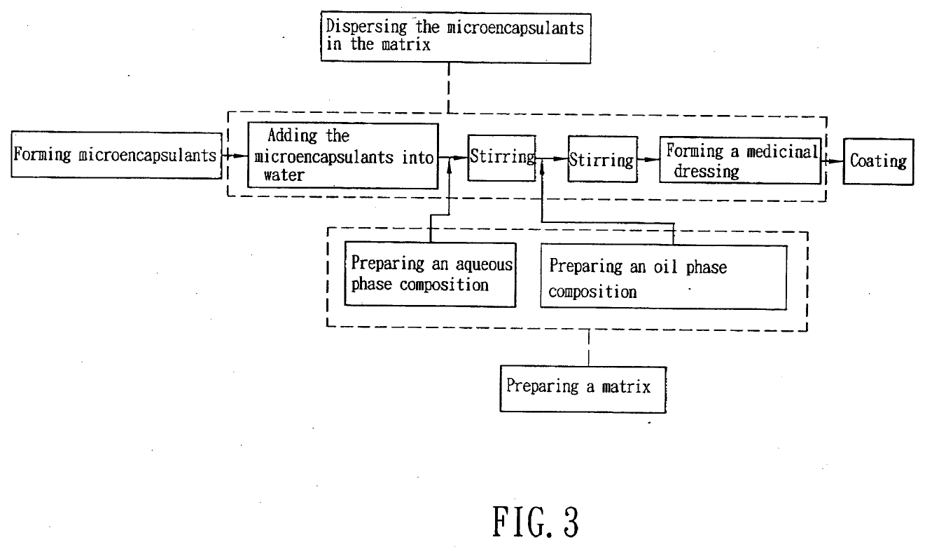

A method for preparing the aforesaid transdermal delivery patch 1 includes the following steps: (A) mixing a first gel, a second gel with a charge opposite to that of the first gel, and a volatile component 142 so as to form the microcapsules 14 enclosing the volatile component 142, the enclosing wall 141 of each of the microcapsules 14 being composed of the first and second gels, (B) preparing the matrix 13; (C) dispersing the microcapsules 14 in the matrix 13 so as to form the medicinal dressing 12; and (D) coating the medicinal dressing 12 on the backing substrate 11 (see FIG. 3).

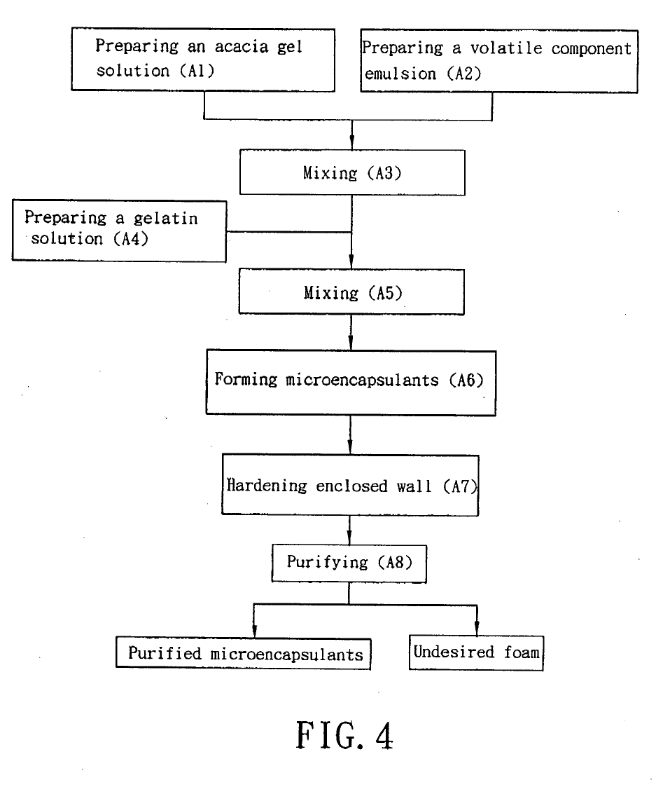

As shown in FIG. 4, the step (A) includes the following sub-steps.

(A1) preparing an acacia gel solution as the first gel by dissolving acacia gel in water, preferably, distilled water, at a temperature higher than gelling point of acacia gel.

(A2) preparing a volatile component emulsion by mixing the volatile component 142 with an emulsifying agent (when menthol is used, it has to be melted before mixing with the emulsifying agent).

(A3) mixing the acacia gel solution with the volatile component emulsion so as to obtain a first mixture.

(A4) preparing a gelatin solution as the second gel by dissolving gelatin in water, preferably, distilled water, at a temperature higher than gelling point of gelatin (about 35° C.) Gelatin can be made from bones, skin, etc. of an animal.

(A5) mixing the first mixture with the gelatin solution to form a second mixture.

(A6) forming the microcapsules 14 by slowly adding an acidic component (e.g., acetic acid) into the second mixture under stirring, followed by adding water with a lower temperature than the second mixture therein so as to form a third mixture, and placing the third mixture in an environment below 10° C. The acidic component is used to change charges of the first and second gels. Before addition of the acidic component, both acacia gel and gelatin particles carry negative charges and cannot attract each other. The acidic component is added to adjust the pH value of the second mixture below isoelectric point of gelatin such that gelatin particles carry positive charges while the charge of acacia gel particles remains the same. By carrying different charges, gelatin and acacia gel particles can attract each other so as to form the microcapsules 14. The volatile component 142 is enclosed in the microcapsules 14 during formation of the latter.

(A7) hardening the enclosing wall 141 of each of the microcapsules 14 by adding an organic solvent (e.g., formaldehyde, used to solidify proteins of gelatin and acacia gel) and then an alkali agent (e.g., sodium hydroxide, used to prevent aggregation of the microcapsules 14) in the third mixture formed in step (A6).

(A8) purifying the microcapsules 14 from the third mixture.

The step (B) of preparing the matrix 13 includes preparations of a first aqueous phase composition, a second aqueous phase composition, and an oil phase composition. The first aqueous phase composition is obtained by mixing Carbopol with water The second aqueous phase composition is obtained by dissolving polyvinyl alcohol and carboxyl methyl cellulose in water. The oil phase composition is obtained by dissolving the volatile component 142 in a lipophilic material (e.g., paraffin wax).

The step (C) of dispersing the microcapsules 14 in the matrix 13 is conducted by adding the microcapsules 14 thus obtained into water followed by adding the first and second aqueous phase compositions. The mixture is stirred for 6 hours. The mixture is well mixed with the oil phase composition so as to form the medicinal dressing 12 consisting of the matrix 13 and the microcapsules 14.

The step (D) is conducted by applying the medicinal dressing 12 onto the backing substrate 11 and subsequently cutting the backing substrate 11 into a desired size.

### EXAMPLE

25 g acacia gel (available from Sigma Company, USA) was dissolved in water and placed in 50° C. water bath, followed by adding therein 5 g menthol (available from Sigma Company, USA), 5 g methyl salicylate (available from Sigma Company, USA), and 3 g span 60 (sorbitan monostearate, available from Merck Company, Germany) so as to form a mixture. 25 g gelatin was dissolved in 60° C. distilled water so as to form a gelatin solution. The gelatin solution thus obtained was slowly added into the mixture, followed by slowly adding 3 ml of 10% acetic acid (pH=4) therein. It is noted that upon adding acetic acid, a plurality of oil beads enclosing the volatile component 142 (i.e., the desired microcapsules 14) were formed so as to form a microcapsule-containing mixture. 200 ml distilled water was added into the microcapsule-containing mixture and stirred in ice bath to allow the temperature to be lower than 10° C. The microcapsule-containing mixture was mixed with 18.5% formaldehyde and stirred for 15 minutes so as to harden the enclosing walls 141 of the microcapsules 14. 2 ml of 20% sodium hydroxide (pH 8-9) was then added, followed by stirring for 0.5 hour and undergoing filtration so as to obtain the microcapsules 14. The microcapsules 14 thus obtained were washed with distilled water to remove the formaldehyde odor.

5 g microcapsules 14 thus obtained was added into 200 ml water and slowly stirred, followed by adding therein an aqueous solution containing a first aqueous phase composition (formed by dissolving 300 g Carbopol 941, available from Merck company, Germany, in water) and a second aqueous phase composition (formed by dissolving 600 g polyvinyl alcohol, available from Merck Company, Germany, and 20 g carboxylmethyl cellulose in 2500 ml water), stirring for 6 hours, and undergoing screening using 60 mesh to remove some undesired precipitates so as to obtain a mixture. An oil phase composition (formed by dissolving an additional volatile component in paraffin wax) was then added into the mixture under stirring so as to obtain a medicinal dressing 12 containing microcapsules 14 having a granular size ranging from 10 to 30 μm (detected using Coulter Laser Particle Counter LS 230, USA, and an optical microscope with an ocular micrometer).

The enclosed percentage of the volatile component 142 ranges from 89.3 to 96.37 wt % based on the total amount of the volatile component 142 used in preparation of the microcapsules 14. The enclosed percentage is determined by the following equation;

(The total amount of the volatile component thus used

in step (A)−the amount of the volatile component remaining

in the distilled water used to remove formaldehyde odor)/the

total amount of the volatile component thus used in

step (A)×100%

A hydrophobic transdermal delivery patch 1 of this invention was prepared using the same procedure except that Carbopol 941, polyvinyl alcohol, and carboxylmethyl cellulose were replaced by natural rubber and styrene-isoprene-styrene block copolymer.

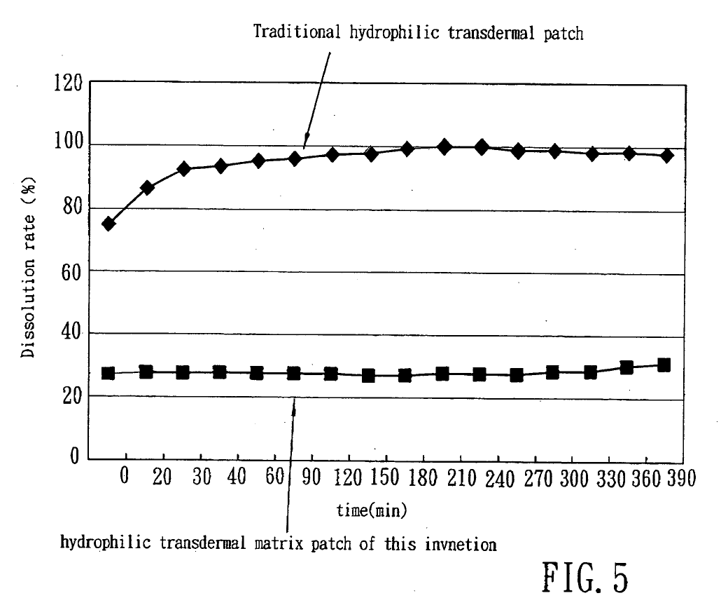

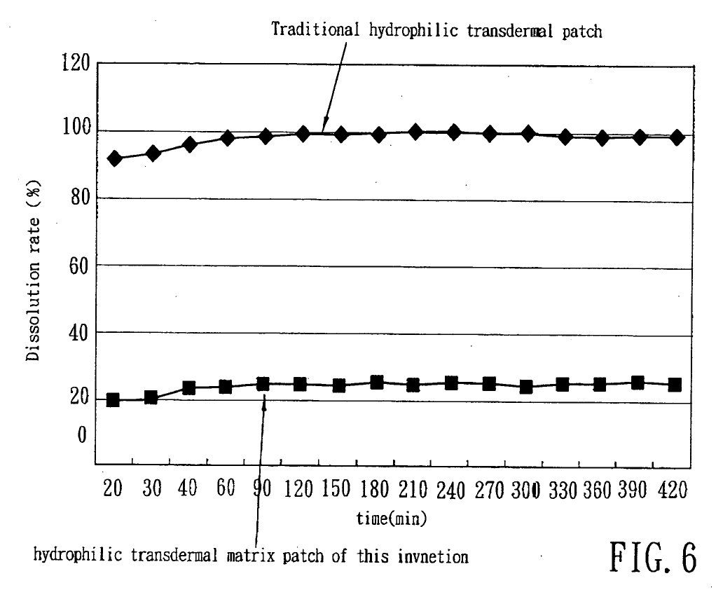

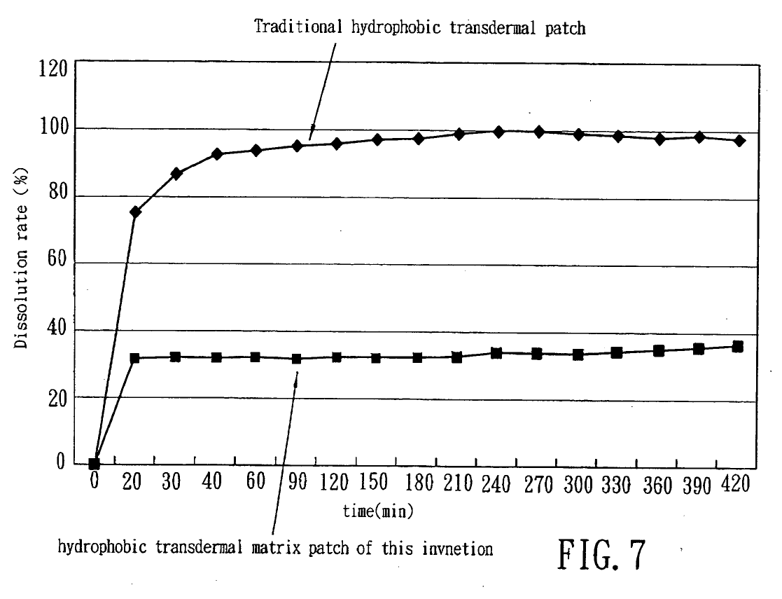

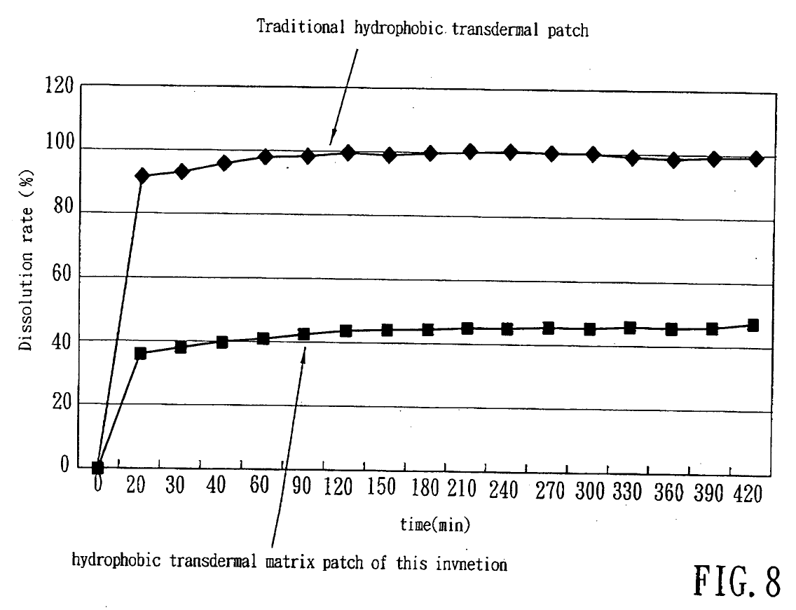

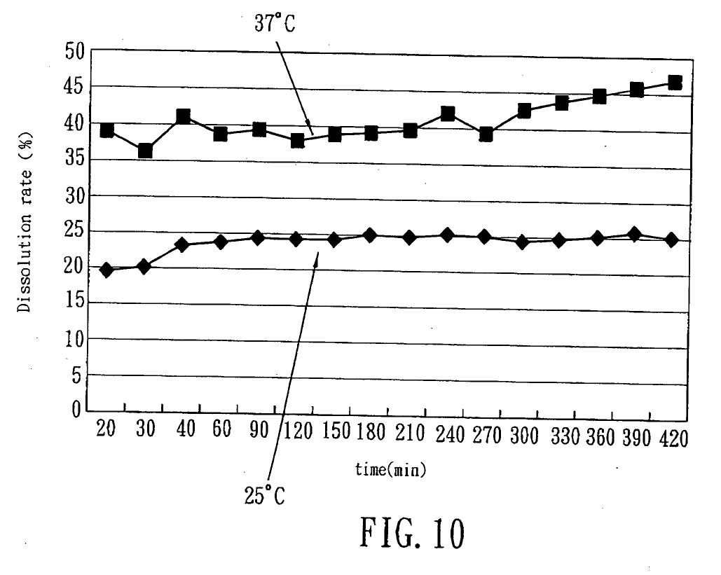

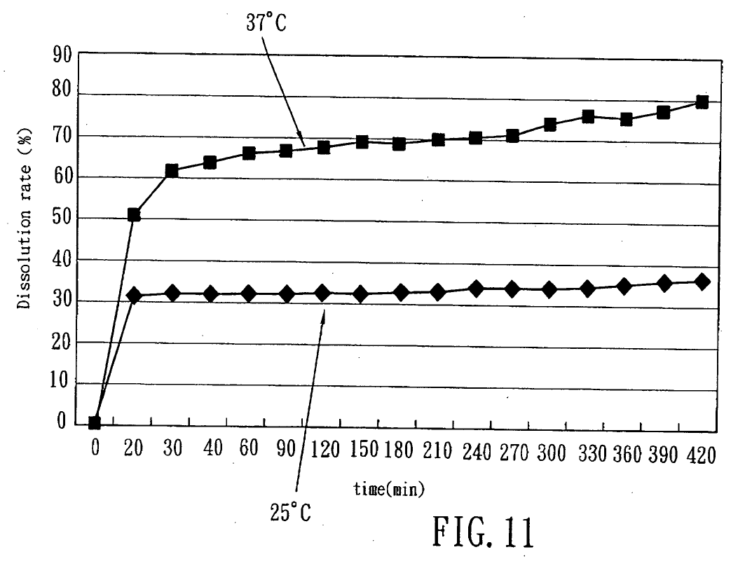

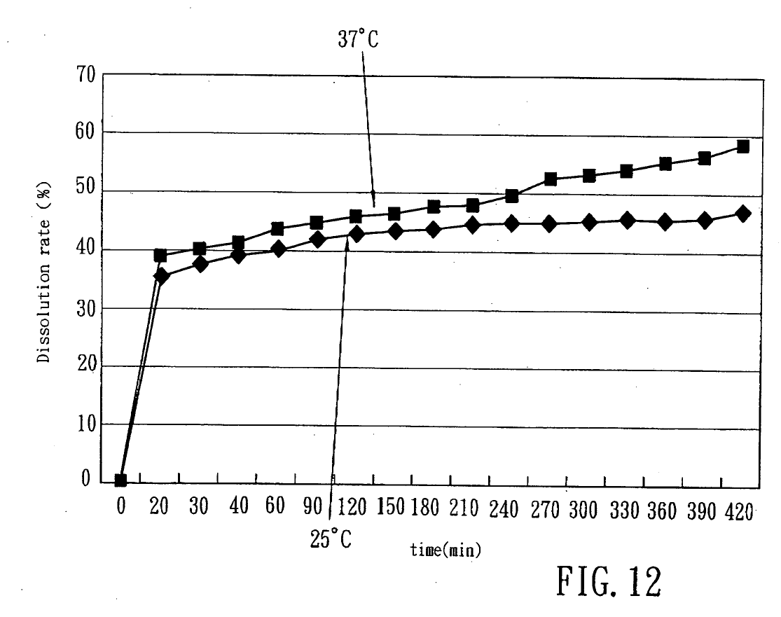

The dissolution rates of menthol and methyl salicylate included in the hydrophilic and hydrophobic transdermal delivery patches 1 were determined using gas chromatography with a flame ionization detector (Shimadzu GC-14A, Japan; dissolution solution: ethanol; rotary speed; 100 rpm; testing temperature: 25 and 37° C.; sampling time: 20, 30, 60, 90, 120, 150, 180, 210, 240, 270, 300, 330, 360, 390, and 420 minutes; sampling amount: 5 ml). The results are shown in FIGS. 5 to 12.

As shown in FIGS. 5 to 8, at 25° C. (i.e., storage temperature), the dissolution rates of menthol and methyl salicylate included in the traditional transdermal patch are higher than those of the hydrophilic and hydrophobic transdermal delivery patches 1 of this invention. This demonstrates that the microcapsules 14 of the transdermal delivery patch 1 of this invention can efficiently prevent the volatile component 142 from vaporizing too fast from the transdermal delivery patch 1 during storage, thereby prolonging the service life of the transdermal delivery patch 1.

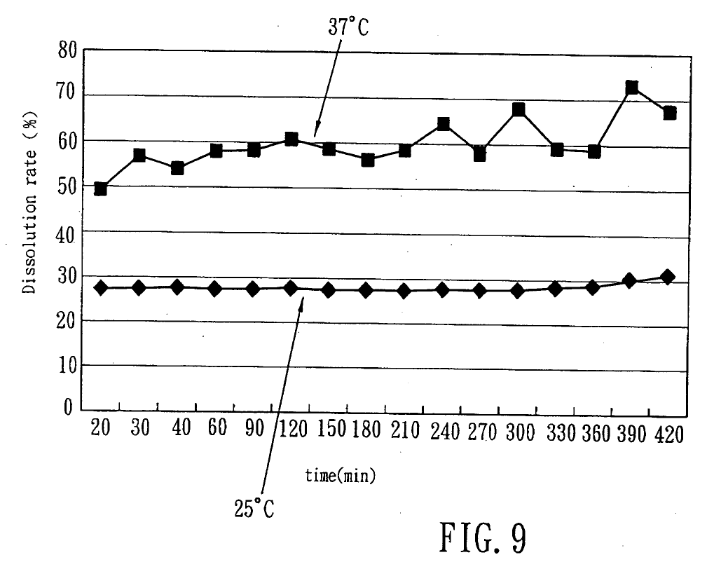

When the transdermal delivery patch 1 of this invention is exposed to a temperature of 37° C. (i.e., applied to skin of a user), the dissolution rates are considerably increased as compared to those at 25° C. (see FIGS. 9 to 12). This shows that the volatile component 142 can also be efficiently released from the transdermal delivery patch 1 of this invention upon application to a user.

With the inclusion of the microcapsules 14 in the transdermal delivery patch 1 of this invention, the dissolution rate of the volatile component 142 can be controlled such that the volatile component 143 can be efficiently retained in the transdermal delivery patch 1 during storage and released from the transdermal delivery patch 1 during use.

While the present invention has been described in connection with what is considered the most practical and preferred embodiments, it is understood that this invention is not limited to the disclosed embodiments but is intended to cover various arrangements included within the spirit and scope of the broadest interpretation and equivalent arrangements.

What is claimed is:

1. A transdermal delivery patch comprising:
a matrix;
a volatile component; and
a plurality of microcapsules dispersed in said matrix, each of said microcapsules having an enclosing wall enclosing said volatile component therein.

2. The transdermal delivery patch of claim 1, wherein each of said microcapsules has a granular size ranging from 10 to 30 μm.

3. The transdermal delivery patch of claim 2, wherein said enclosing wall is composed of acacia gel and gelatin.

4. The transdermal delivery patch of claim 1, wherein said volatile component is selected from the group consisting of menthol, methyl salicylate, and aromatic essential oil of a plant.

5. The transdermal delivery patch of claim 4, wherein said plant is selected from the group consisting of Rhizoma Zingiberis Recens, Fructus Foeniculi, and Flos Caryphylli.

6. The transdermal delivery patch of claim 1, further comprising a backing substrate, said matrix being bonded to said backing substrate.

7. The transdermal delivery patch of claim 1, wherein said matrix is made from a material containing at least one of sodium polyacrylate, potassium polyacrylate, ammonium polyacrylate, monoethanolamine polyacrylate, diethanolamine polyacrylate, triethanolamine polyacrylate, carboxylmethyl cellulose sodium, carboxyl methyl starch, Tragacanth, polyvinyl alcohol, polycaroboxyethylene, polyethylene pyrrolidone, polyacrylic acid, polyethylene glycol, acacia, hydroxypropyl cellulose, hydroxyethyl cellulose, hydroxymethyl cellulose, gelatin, and agar.

8. The transdermal delivery patch of claim 1, wherein said matrix is made from a material containing at least one of synthetic rubber, natural rubber, pine resin, and resin.

9. A method for preparing a transdermal delivery patch, comprising:
mixing a first gel, a second gel with a charge opposite to that of the first gel, and a first volatile component so as to form microcapsules enclosing the first volatile component, each of the microcapsules having an enclosing wall that is composed of the first and second gels;
dispersing the microcapsules in a matrix so as to form a medicinal dressing; and
coating the medicinal dressing on a backing substrate.

10. The method of claim 9, further comprising, during forming of the microcapsules, adding an alkali agent into the mixture of the first and second gels and the first volatile component to prevent aggregation of the microcapsules.

11. The method of claim 9, wherein the matrix is prepared by mixing an aqueous phase composition and an oil phase composition, the aqueous phase composition being made from a material containing at least one of Carbopol, polyvinyl alcohol and carboxylmethyl cellulose, the oil phase composition being made from a material containing a second volatile component and a solvent for dissolving the second volatile component.

12. The method of claim 9, wherein the matrix is prepared by mixing an aqueous phase composition and an oil phase composition, the aqueous phase composition being made from a material containing at least one of synthetic rubber, natural rubber, pine resin, and resin, the oil phase composition being made from a material containing a second volatile component and a solvent for dissolving the second volatile component.

13. The method of claim 9, wherein each of the microcapsules has a granular size ranging from 10 to 30 μm.

14. The method of claim 9, wherein the first gel is acacia gel, and the second gel is gelatin.

15. The method of claim 12, wherein the first volatile component is the same as the second volatile component, and is selected from the group consisting of menthol, methyl salicylate, and aromatic essential oil of a plant.

16. The method of claim 15, wherein the plant is selected from the group consisting of Rhizoma Zingiberis Recens, Fructus Foeniculi, and Flos Caryphylli.
